(19) United States
(12) Patent Application Publication
(10) Pub. No.: US 2020/0172974 A1
(43) Pub. Date: Jun. 4, 2020

(54) BIOMARKER FOR ANTI-TNF THERAPY IN RETINAL DISEASES

(71) Applicant: HADASIT MEDICAL RESEARCH SERVICES & DEVELOPMENT LTD., Jerusalem (IL)

(72) Inventors: Itay CHOWERS, Moshav Beit Zait (IL); Shira HAGBI-LEVI, Jerusalem (IL); Michelle GRUNIN, Jerusalem (IL)

(21) Appl. No.: 16/611,510

(22) PCT Filed: May 8, 2018
(86) PCT No.: PCT/IL2018/050503

§ 371 Date: Nov. 7, 2019

Related U.S. Application Data
(60) Provisional application No. 62/502,862, filed on May 8, 2017.

(51) Int. Cl. C12Q 1/6883; A61K 38/17

(52) U.S. Cl. CPC C12Q 1/6883; A61K 38/1793; A61K 45/06; G01N 2800/164; G01N 2800/52; C12Q 2600/158

(57) ABSTRACT

The present invention is directed to a method for treating nvAMD in a subject having activated macrophages, using a macrophage modulating compound. Further provided are a diagnosis method and a kit for identifying a subject suitable for treatment using the macrophage modulating compound.

### CROSS REFERENCE

This application claims the benefit of priority to U.S. Provisional Patent Application No. 62/502,862 filed May 8, 2017, the contents of which are incorporated herein by reference in their entirety.

### FIELD OF INVENTION

The present invention is in the field of therapy of retinal diseases.

### BACKGROUND OF THE INVENTION

Monocytes, and their monocyte-derived inflammatory macrophages (iMf) descendants were implicated in the pathogenesis of neovascular age related macular degeneration (nvAMD). While it has been recognized that iMfs may be associated with enhanced growth of choroidal neovascularization (CNV), a comprehensive understanding of iMfs' role in the process is lacking. Recently, activated iMfs from nvAMD patients were reported to secrete several pro-angiogenic and pro-inflammatory cytokines, and to accelerate ex-vivo and in-vivo experimental angiogenesis and CNV.

Vascular endothelial growth factor (VEGF) has a major role in the growth of CNV. Accordingly, anti-VEGF compounds are attracting attention as potential compounds for therapy of nvAMD. While application of these compounds results in visual gain and preservation of sight in many nvAMD eyes, patients might also encounter substantial visual loss despite being treated with anti-VEGF based therapies. Indeed, iMfs express VEGF, yet, myeloid-derived VEGF was found to be dispensable for iMfs' contribution to CNV growth. Conceivably, other macrophage-driven cytokines may mediate the pro-angiogenic effect of iMfs in the context of nvAMD. Identifying such cytokines may provide important insights, as well as novel therapeutic targets for nvAMD.

### SUMMARY OF THE INVENTION

In some embodiments, the present invention is directed to a method for treating or attenuating a retinal disease in a subject. Further provided is a method and a kit for identifying a subject afflicted with nvAMD and having activated macrophages as being suitable for treatment using a macrophage modulating compound. In some embodiments, the macrophage compound is a tumor necrosis factor alpha (TNFα) inhibitor.

According to one aspect, there is provided a method for treating a retinal disease in a subject, the method comprising: (i) determining at least one parameter selected from gene expression or factor secretion levels of one or more biomarkers listed under Table 1, in a sample obtained from the subject; and (ii) administering to a subject having an alteration of at least one parameter relative to control, a pharmaceutical composition comprising a therapeutically effective amount of a macrophage modulating compound and at least one pharmaceutically acceptable carrier or diluent.

In some embodiments, the macrophage modulating compound has increased anti-angiogenic activity.

In some embodiments, retinal disease is neovascular age related macular degeneration (nvAMD). In some embodiments, nvAMD comprises choroidal neovascularization (CNV).

In some embodiments, alteration is an increased expression of at least 1.5-fold of a biomarker selected from the group consisting of: FOSB, TMEM176A, TMEM176B, SLED1, CCR2, OLR1 and MOP-1, compared to control, and is indicative of the subject is having a state suitable for treatment by a macrophage modulating compound.

In some embodiments, alteration is a decreased expression of at least 1.5-fold of a biomarker selected from the group consisting of: MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB, compared to control, and is indicative of the subject is having a state suitable for treatment by a macrophage modulating compound.

In some embodiments, the biomarker is selected from the group consisting of: PDGF, TNFα, VEGF, MCP1 (CCL2), and ICAM. In some embodiments, increased secretion of at least 20% of the biomarker, compared to control, is indicative of the subject is having a state suitable for treatment by a macrophage modulating compound.

In some embodiments, the macrophage modulating compound inhibits predominantly activated macrophages. In some embodiments, the macrophage modulating compound is a tumor necrosis factor alpha (TNFα) inhibitor. In some embodiments, the TNFα inhibitor is selected from the group consisting of: nucleic acids, peptides, polypeptides, peptidomimetics, carbohydrates, lipids or other organic or inorganic molecules. In some embodiments, the TNFα inhibitor is selected from the group consisting of: etanercept, infliximab, adalimumab, golimumab and certolizumab.

According to another aspect, there is provided a method of determining a subject afflicted with a retinal disease as suitable for treatment using a macrophage modulating compound, the method comprising: (i) determining at least one parameter associated with activated macrophage in a sample obtained from the subject, the at least one parameter is selected from the group consisting of: gene expression and factor secretion levels; and (ii) determining a significant change in the at least one parameter of at least one biomarker listed under table 1 relatively to control, wherein alteration of at least one parameter relative to control is indicative of the subject is having a state suitable for treatment by the macrophage modulating compound, thereby determining the subject as being suitable for treatment with the macrophage modulating compound.

In some embodiments, the biomarker is selected from the group consisting of: FOSB, TMEM176A, TMEM176B, CCR2, SLED1, OLR1, MOP-1, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA, and IL2RB.

According to another aspect, there is provided a kit for determining macrophage activation in a sample, comprising: at least one molecule that binds to a target biomarker selected from the group consisting of: FOSB, TMEM176A, TMEM176B, CCR2, SLED1, OLR1, MOP-1, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA, IL2RB, PDGF, TNFα, VEGF, MCP1 (CCL2) and ICAM.

In some embodiments, the molecule is selected from the group consisting of: a polynucleotide or a polypeptide. In some embodiments, the polynucleotide hybridizes to the target. In some embodiments, the polypeptide is an antibody.

In some embodiments, the kit is for determining suitability for treatment of a nvAMD disease in a subject by a TNFα inhibitor.

According to another aspect, there is provided a composition comprising a TNFα inhibitor for use in treating a retinal disease in a subject having activated macrophages.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1H demonstrate the effect of cytokines products of M(IFNγ and LPS) iMfs on choroidal sprouting (CSA). (A-F) are fluorescent micrographs of CSA using recombinant cytokines that were previously identified in media from M(IFNγ and LPS) iMfs. Sprouting was observed using a phase microscope. (G) is a vertical bar chart of the sprouting area which was compared across the cytokine-treated wells after normalization to the control well. (H) is a vertical bar chart demonstrating the effect of anti-TNF and anti-VEGF treatment±M(IFNγ and LPS) supernatant. Y-axis indicates the relative sprouting area. *P<0.05 compared with control; +P<0.05 compared with M(IFNγ and LPS) supernatant.

FIGS. 2A-2G demonstrate that anti-TNF therapy abolishes pro-angiogenic effect of M(IFNγ and LPS) iMfs in a LI-CNV model. (A-F) are fluorescent micrographs of CNV areas. All intravitreal injections were performed two days after laser and every two days thereafter for a total of 10 days, while cells, were injected only once, two days after laser injury. (A) PBS-injected rats served as a negative control and (B) M(IFNγ and LPS) treated rats served as a positive control. Anti-TNF and anti-VEGF were tested with (D, F, respectively) or without (C, E, respectively) intravitreal delivery of M(IFNγ and LPS) iMfs (M1) in a rat model of LI-CNV. CNV area was quantified via quantification of isolectin-stained RPE-choroid flat mounts on images obtained by fluorescent microscope (A-F); (G) is a vertical bar chart demonstrating the CNV area which was calculated for each experimental group. The Y-axis presents the averaged±SEM CNV area. *P<0.05 comparing each group to PBS; +P<0.05 comparing to M(IFNγ and LPS) injected rats.

FIGS. 3A-3B are stacked vertical bar graphs demonstrating the evaluation of factors influencing cytokine (A) gene and (B) protein expression levels by iMfs. A mixed designed ANOVA with repeated measures was performed to evaluate the impact of cell origin (dots background); environment (black background); and their interactions (white background) on cytokine gene. Unexplained expression variance is also presented (squared background). Y-axis presents the percentage of variance explained by each factor.

FIG. 4 is a stacked vertical bae graph demonstrating the evaluation of factors influencing iMfs' effect on LI-CNV and CSA. A mixed designed ANOVA with repeated measures was performed to evaluate the impact of cell origin (dots background); environment (black background); and their interactions (white background) on the iMfs' effect in the model of LI-CNV in rats and CSA. Unexplained expression variance is also presented (squared background). Y-axis presents the percentage of variance explained by each factor.

### DETAILED DESCRIPTION OF THE INVENTION

In some embodiments, the present invention is directed to a method for treating or attenuating a retinal disease in a subject. Further provided is a method and a kit for identifying a subject afflicted with nvAMD and having activated macrophages as being suitable for treatment using a macrophage modulating compound.

### Method of Treatment

In some embodiments, the present invention is directed to a method for treating or attenuating a retinal disease in a subject, the method comprising: (i) selecting a subject having activated macrophages; and (ii) administering to the subject a pharmaceutical composition comprising a therapeutically effective amount of a macrophage-modulating compound having increased anti-angiogenic activity and at least one pharmaceutically acceptable carrier or diluent.

As used herein, the term “retinal disease” encompasses any disease of the retina, i.e., the light-sensitive membrane at the back of the eye. The terms “retinal disease” and “retinopathy” are used herein interchangeably.

In some embodiments, retinopathy comprises proliferative or non-proliferative retinopathy. Non-limiting examples of non-proliferative retinopathy include, but are not limited to: hypertensive retinopathy, retinopathy of prematurity, radiation retinopathy, solar retinopathy, and retinopathy associated with sickle cell disease. Non-limiting example of a proliferative retinopathy comprises diabetic retinopathy.

Age-related macular degeneration (AMD) is the most common cause of irreversible central vision loss in elderly patients. In one embodiment, AMD is dry AMD. In some embodiments, AMD is wet AMD. In one embodiment, AMD in neovascularization AMD (nvAMD). In some embodiments, AMD is diagnosed according to any method known to one of ordinary skill in the art. Non-limiting examples for diagnosing AMD include: funduscopic examination, color fundus photography, fluorescein angiography, optical coherence tomography and others.

In some embodiments, wet AMD comprises any condition in which new abnormal blood vessels develop under the retina. In some embodiments, abnormal blood vessels developments are referred to as “choroidal neovascularization” (CNV). In some embodiments, localized macular edema or hemorrhage induce elevation of the macula or cause a localized retinal pigment epithelial detachment. In some embodiments, neovascularization causes a disciform scar under the macula.

In some embodiments, method of the present invention is directed to treating nvAMD in a subject having activated macrophages. As defined herein, the term “macrophage” refers to the largest type of a white blood cell. In one embodiment, a blood-circulating macrophage is a monocyte. In one embodiment, a macrophage is a monocyte which has migrated from the blood stream into any tissue of the body. In some embodiments, the term “M1 macrophage” refers to a macrophage which promotes inflammatory response. In some embodiments, an M1 macrophage is pro-angiogenic. In some embodiments, M1 macrophage is a classically activated macrophage. In some embodiments, M1 macrophage is different from M0 (i.e., undifferentiated) or M2 (i.e., alternatively activated) macrophage and is distinguished using any method known to one of ordinary skill in the art. Non-limiting examples include flow cytometry or immunohistochemical staining based on M1 macrophage specific biomarkers, including but not limited to: CD14, CD40, CD11b, CD64, F4/80/EMR1 (murine/human), lysozyme M, MAC-1/MAC-3, CD68, and others.

In some embodiments, methods of the present invention are directed to treating nvAMD in a subject having activated macrophages, either classically activated or alternatively activated macrophages.

The terms “activated macrophage” and “M1 macrophage” are used herein interchangeably.

In some embodiments, methods of the present invention are directed to identifying and treating a retinal disease in a subject. In some embodiments, methods of the present invention improve a subject's reaction to treatment. In some embodiments, treatment of a retinal disease in a subject initially identified as having retinal disease has higher efficacy. The term “higher efficacy” used herein, is interchangeable with “better”, “improved” or any other synonym thereof. In some embodiments, a subject pre-identified as having a retinal disease and then treated for the retinal disease, has higher recovery rates compared to a subject not pre-identified for the retinal disease. In some embodiments, higher as used herein is an increase of at least 5%, at least 10%, at least 20%, at least 35%, at least 50%, at least 65%, at least 70%, at least 80%, at least 90% or at least 99%. In some embodiments, higher as used herein is an increase of 1-5%, 4-8%, 7-10%, 9-20%, 15-30%, 25-40%, 35-50%, 45-60%, 55-70%, 65-80%, 75-90%, 85-90%, 90-99%, or 95-100%. In some embodiments, higher as used herein is an increase of at least 2-fold, at least 3-fold, at least 4-fold, at least 5-fold, at least 6-fold, at least 7-fold, at least 8-fold, at least 9-fold, or at least 10-fold. Each possibility represents a separate embodiment of the present invention.

In some embodiments, as defined herein, the term “angiogenesis” refers to the formation and development of blood vessels. As used herein, the term “pro-angiogenic” comprises any one of a molecule, a macromolecule or a process promoting angiogenesis. The terms “angiogenic” and “pro-angiogenic” are used herein interchangeably.

As used herein, the present invention is directed to a method and compositions comprising macrophage modulating compound(s). In one embodiment, a macrophage modulating compound(s) has an increased anti-angiogenic activity. In one embodiment, increased anti-angiogenic activity is increased inhibition of angiogenesis. In one embodiment, the method of the disclosed invention is directed to inhibition of pro-angiogenesis effect of activated M1 macrophages, wherein inhibiting is reducing angiogenesis by more than 2%, by more than 5%, by more than 10%, by more than 25%, by more than 50%, by more than 75%, by more than 90%, by more than 95%, or by more than 99%. In some embodiments, the method of the disclosed invention is directed to inhibition of pro-angiogenesis effect of activated M1 macrophages, wherein inhibiting is reducing angiogenesis by 1-2%, 1.5-5%, 4-10%, 12-25%, 20-50%, 45-75%, 70-90%, 75-95%, or 90-100%. Each possibility represents a separate embodiment of the present invention. As would be apparent to the skilled artisan, angiogenesis can be detected according to any method known in the art. Non-limiting examples include: choroidal sprouting assay (CSA) and laser induced choroidal neovascularization (LI-CNV) assay. In one embodiment, angiogenesis can be assayed in vivo, in vitro or ex vivo.

As used herein, the terms “attenuate”, “inhibit”, “revert”, and “reverse” are interchangeable.

In another embodiment, the macrophage modulating compound or a composition comprising thereof is for use in treatment, amelioration, reduction, and/or prevention of a retinal disease or condition in a subject in need thereof. In some embodiments, there is provided a composition comprising an effective amount of macrophage modulating compound for use in the treatment or prevention of a retinal disease or condition in a subject in need thereof.

In some embodiments, there is provided a use of a composition comprising an effective amount of the macrophage modulating compound in the preparation of a medicament for the treatment, amelioration, reduction, or prevention of a disease associated with retinal degeneration in a subject in need thereof.

In one embodiment, the macrophage modulating compound is provided to the subject per se. In one embodiment, one or more of the macrophage modulating compounds are provided to the subject per se. In one embodiment, the macrophage modulating compound is provided to the subject as part of a pharmaceutical composition where it is mixed with a pharmaceutically acceptable carrier. In one embodiment, one or more of the macrophage modulating compounds are provided to the subject as part of a pharmaceutical composition where they are mixed with a pharmaceutically acceptable carrier.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with and/or at risk of being afflicted with a condition associated with increased cell proliferation.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compounds prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

### Method and Kit for Diagnosis

In some embodiments, the invention is directed to a method and a kit for identifying a subject afflicted by a nvAMD disease or condition for being suitable for treatment using a macrophage modulating compound. In some embodiments, the methods and kit of the invention are directed to determining the suitability of a subject for a treatment using a TNFα inhibitor.

In some embodiments, method of the present invention is directed to determining at least one parameter associated with activated macrophage in a sample obtained from a subject. In some embodiments, the parameter is selected from the group consisting of: gene expression and factor secretion levels. In some embodiments, methods of the present invention are directed to determining a significant change of a parameter relatively to control. In some embodiments, a significant change or alteration of a parameter relative to control is indicative of the subject is having a state suitable for treatment by a macrophage modulating compound.

As defined herein “biological sample” refers to a physical specimen from any animal. In another embodiment, biological sample is obtained from a mammal. In another embodiment, biological sample is obtained from a human. In another embodiment, biological sample is obtained well within the capabilities of those skilled in the art. The biological sample includes, but not limited to, biological fluids such as serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, and tissue culture media, including tissue extracts such as homogenized tissue, and cellular extracts. In another embodiment, a biological sample is a biopsy. In another embodiment, a biological sample is a resected tumor. In another embodiment, a biological sample includes histological sections processed as known by one skilled in the art. The terms “sample” and “biological sample” used herein, are interchangeable.

As defined herein, the term “baseline level” and “control” are interchangeable and refer to a gene expression and factor secretion levels of an M1 macrophage measured in the subject before or at early nvAMD. In one embodiment, before is at least 1 week, at least 1 month, at least 3 months, at least 6 months, at least 9 months or at least 12 months before or at early nvAMD. Each possibility represents a separate embodiment of the present invention. In another embodiment, a gene expression and factor secretion levels of an M1 macrophage in a subject afflicted with nvAMD is measured compared to a non-afflicted cell or tissue obtained from the same subject. In one embodiment, a gene expression and factor secretion levels of an M1 macrophage in a subject afflicted with nvAMD is measured compared to a nvAMD-non-afflicted control subject. In some embodiments, a gene expression and factor secretion levels of an M1 macrophage in a subject afflicted with nvAMD is measured compared to a cell line. In one embodiment, a cell is selected form human-derived cell line or non-human-derived cell line.

In another aspect, the present invention is directed to a method of diagnosis assaying a plurality of mRNAs expression levels in a sample obtained from a subject. In some embodiments, the present invention is directed to a diagnostic kit comprising means for determining the expression level of a plurality of mRNAs selected from the group consisting of: SLED1, FOSB, OLR1, MOP-1, TMEM176A, TMEM176B, MS4A1, CD3 G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB. As used herein, the terms “mRNA expression” and “gene expression” are interchangeable.

In another aspect, the present invention is directed to a method of diagnosis assaying a plurality of factors secreted levels in a sample obtained from a subject. In some embodiments, the present invention is directed to a diagnostic kit comprising means for determining the secreted levels of a plurality of factors selected from the group consisting of: PDGF, TNFα, VEGF, MCP1 (CCL2), and ICAM.

As defined herein, a significant change or alteration of gene expression or factor secreted levels relative to control is indicative of the subject is having a state suitable for treatment by a macrophage modulating compound. In some embodiments, a significant change or alteration of gene expression comprises increase i.e., over expression or decrease i.e., down-regulation of specific genes, as described herein below. In some embodiments, a significant change or alteration of factor secreted levels comprises increased or decreased secretion of specific factors, as described herein below. As defined herein, a significant change or alteration of gene expression or factor secreted levels relative to control that is indicative of the subject is having a state suitable for treatment by a macrophage modulating compound is by at least 1.1-fold, by at least 1.5-fold, by at least 2-fold, by at least 3-fold, by at least 4-fold, by at least 5-fold, by at least 6-fold, by at least 7-fold, by at least 8-fold, by at least 9-fold or by at least 10-fold. In some embodiments, a significant change or alteration of gene expression or factor secreted levels relative to control that is indicative of the subject is having a state suitable for treatment by a macrophage modulating compound is by 1.1-1.5-fold, 1.3-2-fold, 2-3-fold, 2.5-4-fold, 3-5-fold, 4-6-fold, 5-7-fold, 5.5-8-fold, 7-9-fold or by 6-10-fold. In some embodiments, a significant change or alteration of gene expression or factor secreted levels relative to control that is indicative of the subject is having a state suitable for treatment by a macrophage modulating compound is by 5-10%, 7-15%, 12-20%, 17-35%, 30-45%, 40-60%, 50-70%, 65-85%, 80-95% or 90-100%. In some embodiments, a significant change or alteration of gene expression or factor secreted levels relative to control that is indicative of the subject is having a state suitable for treatment by a macrophage modulating compound is by at least 10%, by at least 20%, by at least 30%, by at least 50%, by at least 75%, by at least 100%, by at least 150%, by at least 200%, by at least 250%, by at least 300%, by at least 350%, by at least 400%, by at least 450% or by at least 500%. Each of the possibilities represents a separate embodiment of the present invention. In one embodiment, a gene expression fold change is calculated as a binary logarithm. In one embodiment, a binary logarithm is a base 2 logarithm i.e., Log 2.

In some embodiments, over-expressed or down-regulated genes which are indicative of a subject suitability for treatment using macrophage modulating compound are selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB.

In some embodiments, over-expressed genes which are indicative of a subject suitability for treatment using macrophage modulating compound are selected from the group consisting of: SLED1, FOSB, OLR1, MOP-1, TMEM176A, and TMEM176B.

In some embodiments, down-regulated genes which are indicative of a subject suitability for treatment using macrophage modulating compound are selected from the group consisting of MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB.

According to another embodiment, the invention is directed to a kit comprising at least two probes complementary to a plurality of mRNAs selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB.

According to another embodiment, the invention provides a kit comprising at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, or at least 79 probes complementary to a plurality of mRNAs selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB.

In one embodiment, the composition for use in diagnosis comprises at least two complementary molecules to the mRNAs selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB. In one embodiment the composition for use in diagnosis comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, or at least 79 probes complementary to a plurality of mRNAs selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB.

As non-limiting examples, a subject afflicted with nvAMD would be considered suitable for treatment using a macrophage modulating compound according to methods of diagnosis and kit of the present invention, when the expression of at least one gene or mRNA thereof, selected from the group consisting of: SLED1, FOSB, OLR1, MOP-1, TMEM176A, TMEM176B, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB, is over-expressed or down-regulated by more than 1.1, 1.2, 1.5, 1.75, 1.9, 2, 2.2, 2.4, or 2.5-fold compared to a control baseline. Each possibility represents a separate embodiment of the present invention.

As non-limiting examples, a subject afflicted with nvAMD would be considered suitable for treatment using a macrophage modulating compound according to methods of diagnosis and kit of the present invention, when the secretion levels of a at least one factor, selected from the group consisting of: PDGF, TNFα, VEGF, MCP1 (CCL2), and ICAM, is increased or decreased by at least 10%, at least 15%, at least 20%, or at least 25% compared to a control baseline. Each possibility represents a separate embodiment of the present invention.

In another embodiment, the kit comprises mRNA hybridization or amplification reagents; and at least one probe or amplification primer specific for each member selected from the plurality of mRNAs. In some embodiments, the kit further comprises means for collecting a sample (e.g., blood, biopsy, etc.) from a subject. In another embodiment, the diagnostic kit further comprises instructions for performing the necessary steps for determining mRNAs expression levels, e.g., in a sample obtained from a subject.

### Hybridization Assays

Detection of a nucleic acid of interest in a biological sample (e.g., mRNA) may optionally be affected by hybridization-based assays using an oligonucleotide probe. Traditional hybridization assays include PCR, reverse-transcriptase PCR, real-time PCR, RNase protection, in-situ hybridization, primer extension, dot or slot blots (RNA), and Northern blots (i.e., for RNA detection). More recently, PNAs have been described (Nielsen et al. 1999, Current Opin. Biotechnol. 10:71-75). other detection methods include kits containing probes on a dipstick setup and the like.

The term “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using the probe under sufficiently stringent hybridization conditions permits the selective detection of a specific target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support or the like, as sites for hybridization of other oligonucleotides, as restriction enzymes sites or binding sites for other nucleic acid binding enzymes, etc. In certain embodiments, a probe of the invention is included in a nucleic acid that comprises one or more labels (e.g., a reporter dye, a quencher moiety, a fluorescent labeling, etc.), such as a 5′-nuclease probe, a FRET probe, a molecular beacon, or the like, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable duplexes may contain mismatched bases or unmatched bases. Modification of the stringent conditions may be necessary to permit a stable hybridization duplex with one or more base pair mismatches or unmatched bases. Sambrook et al., Molecular Cloning: A Laboratory Manual, 3rd Ed., Cold Spring Harbor Laboratory Press, Cold Spring Harbor, N.Y. (2001), which is incorporated by reference, provides guidance for suitable modification. Stability of the target/probe duplex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids. Exemplary probe nucleic acids include 5′-nuclease probes, molecular beacons, among many others known to persons of skill in the art.

As used herein, “hybridization” refers to a reaction in which at least one polynucleotide reacts to form a complex that is stabilized via hydrogen bonding between the bases of the nucleotide residues. The hydrogen bonding may occur by ‘Watson-Crick base pairing’, in any other sequence-specific manner. A hybridization reaction may constitute a step in a more extensive process, such as the initiation of a Polymerase chain reaction (PCR).

Hybridization reactions can be performed under conditions of different stringency. Under stringent conditions, nucleic acid molecules at least 60%, at least 65%, at least 70%, at least 75% or identical to each other remain hybridized to each other. A non-limiting example of highly stringent hybridization conditions is hybridization in 6× Sodium chloride/Sodium citrate (SSC) at approximately 45° C., followed by one or more washes in 0.2×SSC and 0.1% SDS at 50° C., at 55° C., at about 60° C., or more.

When hybridization occurs in an anti-parallel configuration between two single-stranded polynucleotides, those polynucleotides are described as complementary.

Hybridization based assays which allow the detection of a biomarker of interest in a biological sample rely on the use of probe(s) which can be 10, 15, 20, or 30 to 100 nucleotides long, optionally from 10 to 50, or from 40 to 50 nucleotides long.

Thus, the polynucleotides of the biomarkers of the invention, according to at least some embodiments, are optionally hybridizable with any of the herein described nucleic acid sequences under moderate to stringent hybridization conditions.

The detection of hybrid duplexes can be carried out by several methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well-known methods. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides according to at least some embodiments of the present invention can be labeled subsequently to synthesis, by incorporating biotinylated dNTPs or rNTPs, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others (e.g., Kricka et al. (1992), Academic Press San Diego, Calif.) can be attached to the oligonucleotides. Preferably, detection of the biomarkers of the invention is achieved by using TaqMan assays, preferably by using combined reporter and quencher molecules (Roche Molecular Systems Inc.).

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well-known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target polynucleotide or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNase A prior to hybridization, to assess false hybridization.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA), deoxyribonucleic acid (DNA) or locked nucleic acid (LNA).

### Fluorescent In-Situ Hybridization (FISH)

An additional nucleic acid test (NAT) test known in the art is fluorescence in situ hybridization (FISH). FISH uses fluorescent single-stranded DNA or RNA probes which are complementary to the nucleotide sequences that are under examination (genes, chromosomes or RNA). These probes hybridize with the complementary nucleotide and allow the identification of the chromosomal location of genomic sequences of DNA or RNA.

Detection of a nucleic acid of interest in a biological sample may also optionally be affected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions. Although other primer nucleic acid lengths are optionally utilized, they typically comprise hybridizing regions that range from about 8 to about 100 nucleotides in length. Short primer nucleic acids generally utilize cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled (e.g., a SCORPION primer, etc.), if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or otherwise known in the art. One of skill in the art will recognize that, in certain embodiments, primer nucleic acids can also be used as probe nucleic acids.

Amplification of a selected, or target, nucleic acid sequence may be carried out by a number of suitable methods (e.g., Kwoh et al., 1990, Am. Biotechnol. Lab. 8:14). Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides according to at least some embodiments of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In some embodiments, amplification of a nucleic acid sample from a subject is amplified under conditions which favor the amplification of the most abundant differentially expressed nucleic acid. In one embodiment, RT-PCR is carried out on an RNA sample from a subject under conditions which favor the amplification of the desired RNA. In one embodiment, the desired RNA is a plurality of RNAs. In another embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (e.g., mRNA) for practicing the present invention may be obtained according to well-known methods.

Oligonucleotide primers according to at least some embodiments of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

### Polymerase Chain Reaction (PCR)

The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified”.

The PCR and other nucleic acid amplification reactions are well known in the art. In one embodiment, the pair of oligonucleotides according to this aspect of the present invention are selected to have compatible melting temperatures (Tm), e.g., melting temperatures which differ by less than that 7° C., by less than 5° C., by less than 4° C., or by less than 3° C. In one embodiment, the pair of oligonucleotides according to this aspect of the present invention are selected to have compatible Tm which differ by less than that 5-7° C., 3-5° C., 2-4° C., 1-3° C. or 0-1° C. Each possibility represents a separate embodiment of the present invention.

The skilled artisan will understand that other PCR related methods may be used alone or combined. Non-limiting exemplary method are described herein below.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

In another aspect, the present invention is directed to a kit and a method suitable for assaying a plurality of proteins in a sample obtained from a subject.

In some embodiments, the present invention is directed to a diagnostic kit comprising means for determining at least one protein or polypeptide level of a plurality of proteins or polypeptides selected from the group consisting of: PDGF, TNFα, VEGF, MCP1 (CCL2), and ICAM. In some embodiments, the present invention is directed to a diagnostic kit comprising means for determining at least 2, at least 3, at least 4, or at least 5 proteins or polypeptides level of a plurality of proteins or polypeptides selected from the group consisting of: PDGF, TNFα, VEGF, MCP1 (CCL2), and ICAM.

In some embodiments, the expression, and the level of expression, of proteins or polypeptides of interest can be detected through immunohistochemical staining of tissue slices or sections. Additionally, proteins/polypeptides of interest may be detected by western blotting, enzyme-linked immunosorbent assay (ELISA) or Radioimmunoassay (MA), employing protein-specific antibodies.

### Compositions

According to another aspect, the present invention provides a pharmaceutical composition comprising as an active ingredient a therapeutically effective amount of macrophage modulating compound having increased anti-angiogenic activity, and a pharmaceutically acceptable carrier and/or diluent. In some embodiments, the pharmaceutical composition facilitates administration of a macrophage modulating compound to the target tissue.

In some embodiments, macrophage modulating compound comprises Tumor necrosis factor α (TNFα) inhibitor(s). In some embodiments, the term “TNFα inhibitor” as used herein, refers to any molecule that acts with specificity to reduce TNFα activity, e.g., by blocking receptor(s) binding or by reducing expression of the TNFα gene. In some embodiments, a TNFα inhibitor is selected from the group consisting of: nucleic acids, peptides, polypeptides, peptidomimetics, carbohydrates, lipids and other organic or inorganic molecules.

In one embodiment, TNFα inhibitor is a soluble protein. In one embodiment, TNFα inhibitor is an insoluble protein. In one embodiment, TNFα inhibitor is a membrane anchored protein. In one embodiment, TNFα inhibitor is a polypeptide comprising a soluble TNFα receptor (TNFR) polypeptide fragment that binds to TNFα. In one embodiment, TNFR is selected from the group consisting of: TNFR1 and TNFR2. In one embodiment, TNFα inhibitor is a protease. In one embodiment, TNFα inhibitor is an antibody. In one embodiment, TNFα inhibitor is a polypeptide comprising an antigen binding fragment of an anti-TNFα antibody. In one embodiment, TNFα inhibitor is a molecule of the extracellular matrix. In one embodiment, TNFα inhibitor is a proteoglycan. In one embodiment, TNFα inhibitor is a polynucleotide. In one embodiment, TNFα inhibitor is an anti-sense polynucleotide. In one embodiment, TNFα inhibitor is a regulatory RNA. In one embodiment, TNFα inhibitor is a short-interfering RNA (siRNA). In one embodiment, TNFα inhibitor is a microRNA (miRNA). In some embodiments, a TNFα inhibitory polynucleotide reduces TNFα gene expression levels, e.g., mRNA levels. In some embodiments, mRNA levels of the TNFα encoding gene are reduced by TNFα gene editing. In some embodiments, gene editing is molecular alteration in the TNFα genomic polynucleotide sequence resulting in reduction of the TNFα gene expression levels. In some embodiments, gene editing is achieved by Clustered Regularly Interspaced Short Palindromic Repeats (CRISPR) system. In one embodiment, TNFα inhibitor is a molecule capable of irreversible binding of TNFα. In one embodiment, TNFα inhibitor is a molecule capable of reversible binding of TNFα. In one embodiment, TNFα inhibitor is a molecule capable of binding TNFα with an affinity equal to-or greater than TNFR. In one embodiment, TNFα inhibitor is a molecule capable of binding TNFα with affinity comparable to TNFR. In one embodiment TNFα inhibitor is a molecule binding TNFα with a dissociation constant (K_{d}) of 10⁻¹⁰ M or greater. In another embodiment, TNFα inhibitor is any small molecule capable of inhibiting TNFα signaling. In some embodiments, a TNFα inhibitor is selected from the group consisting of: etanercept, infliximab, adalimumab, golimumab and certolizumab.

As used herein, a “peptide” refers to either a naturally or artificially manufactured short chain of amino acid monomers, which are linked to one another by means of amide (peptide) bonds. With this respect, a “polypeptide” is a long, continuous peptide polymer. Peptides and polypeptides may comprise 50 amino acids, 40 amino acids, 30 amino acids, 20 amino acids, or less than 10 amino acids. The terms “peptide”, “polypeptide” and “protein” used herein, are interchangeable.

“Peptide mimetics” or “peptidomimetics” are structures which serve as substitutes for peptides in interactions between molecules (Morgan et al., 1989). Peptide mimetics include synthetic structures which may or may not contain amino acids and/or peptide bonds but retain the structural and functional features of a peptide, or agonist or antagonist (i.e. enhancer or inhibitor) of the invention. Peptide mimetics also include peptoids, oligopeptoids (Simon et al., 1972); and peptide libraries containing peptides of a designed length representing all possible sequences of amino acids corresponding to a motif, peptide, or agonist or antagonist (i.e. enhancer or inhibitor) of the invention.

In some embodiments, compositions of the present invention are directed to modulating activity of predominantly M1 macrophages. In some embodiments, compositions of the present invention comprise TNFα inhibitor(s), modulating activity of predominantly M1 macrophages. In one embodiment, TNFα is predominantly expressed or produced by activated M1 macrophages.

By another aspect, there is provided use of the composition of the present invention for preparation of a medicament for treating age-related ophthalmic disease.

As used herein, the term “carrier”, “adjuvant” or “excipient” refers to any component of a pharmaceutical composition that is not the macrophage modulating compound and has no anti-angiogenic activity.

As used herein, the term “pharmaceutically acceptable” means suitable for administration to a subject, e.g., a human and/or for a proliferating cell as described herein. For example, the term “pharmaceutically acceptable” can mean approved by a regulatory agency of the Federal or a state government or listed in the U. S. Pharmacopeia or other generally recognized pharmacopeia for use in animals, and more particularly in humans. In some embodiments, pharmaceutically acceptable carrier is non-toxic, inert solid, semi-solid liquid filler, diluent, encapsulating material, formulation auxiliary of any type, or simply a sterile aqueous medium, such as saline. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents that may be useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

According to an embodiment of the invention, pharmaceutical compositions contain 0.1%-95% of macrophage modulating compound. According to another embodiment of the invention, pharmaceutical compositions contain 1-70% macrophage modulating compound. According to another embodiment of the invention, the composition or formulation to be administered may contain a quantity of macrophage modulating compounds, according to embodiments of the invention in an amount effective to treat the condition or disease of the subject being treated.

According to one embodiment, the compositions of the present invention are administered in the form of a pharmaceutical composition comprising at least one of the active components of this invention (macrophage modulating compound having increased anti-angiogenic activity) together with a pharmaceutically acceptable carrier or diluent. In another embodiment, the compositions of this invention can be administered either individually or together in any conventional sub-retinal dosage form.

The carrier may comprise, in total, from about 0.1% to about 99.99999% by weight of the pharmaceutical compositions presented herein.

The compositions also include incorporation of the active material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

As used herein, the terms “administering,” “administration,” and like terms refer to any method which, in sound medical practice, delivers a composition containing an active agent to a subject in such a manner as to provide a therapeutic effect.

In one embodiment, depending on the severity and responsiveness of the condition to be treated, dosing can be of a single or a plurality of administrations, with course of treatment lasting from several days to several weeks or until cure is affected or diminution of the disease state is achieved.

The dose will vary depending on the subject and upon the particular route of administration used. Commercially available assays may be employed to determine optimal dose ranges and/or schedules for administration. Effective doses may be extrapolated from dose-response curves obtained from animal models.

As used herein, the term “therapeutically active molecule” or “therapeutic agent” means a molecule, group of molecules, complex or substance administered to an organism for diagnostic, therapeutic, preventative medical, or veterinary purposes. This term includes pharmaceuticals, e.g., small molecules, treatments, remedies, biologics, devices, and diagnostics, including preparations useful in clinical screening, prevention, prophylaxis, healing, imaging, therapy, surgery, monitoring, and the like. This term can also specifically include nucleic acids and compounds comprising nucleic acids that produce a bioactive effect, for example.

The term “therapeutically effective amount” refers to the concentration of macrophage modulating compound(s) having increased anti-angiogenic activity, or their combination, normalized to body weight, that is effective to treat a disease or disorder in a mammal. The term “a therapeutically effective amount” refers to an amount effective, at dosages and for periods of time necessary, to achieve the desired therapeutic or prophylactic result. A physician or veterinarian of ordinary skill can readily determine and prescribe the effective amount of the bioactive agent required.

In some embodiments, a composition of the invention comprises pharmaceutically active agents. In some embodiments, pharmaceutically active agents are added prior to transplantation. Pharmaceutically active agents include but are not limited to any of the specific examples disclosed herein. Those of ordinary skill in the art will recognize also numerous other compounds that fall within this category and are useful according to the invention.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Patients Eligibility and Exclusion Criteria

Neovascular age-related macular degeneration (nvAMD) patients (n=2 males; age: 92 and 86 years) were recruited from the retina clinic of the Department of Ophthalmology at the Hadassah-Hebrew University Medical Center. Diagnosis of AMD was based on AREDS criteria, and CNV was confirmed via fluorescein angiogram and optical coherence tomography. Lesions comprised at least 50% active CNV and less than 25% sub-retinal hemorrhage. There were no other retinal diseases in the eyes included in the study. Specifically, other potential causes for CNV such as myopia, trauma, or uveitis were excluded. Also excluded were patients with a major systemic illness, such as cancer, autoimmune disease, congestive heart failure, or uncontrolled diabetes. The patients signed an informed consent form, and the study was approved by the institutional ethics committee.

### Macrophage Preparation

Blood samples (30 ml) were collected from nvAMD patients in EDTA tubes (BD Bioscience). Monocytes were isolated from whole blood, differentiated into iMfs (M0), and activated into M(IFNγ and LPS; M1-like iMfs), as previously described (Hagbi-Levi et al., (2016)). This macrophage subtype was chosen as it leads to enhanced neovascularization in vitro and in-vivo. Medium of the polarized iMfs' cell culture was collected and kept in −20° C. for use in the choroidal sprouting assays (CSA). iMfs were collected with 0.025% trypsin (TriReagent; Sigma-Aldrich, Munich, Germany) washed with RPMI+FCS, following 3 washes with PBS and immediately used for the in-vivo experiments.

### Choroid-Sprouting Assay (CSA)

An ex-vivo angiogenesis assay was performed to assess the angiogenic effect of candidate cytokines that were upregulated in the supernatant of M(IFNγ and LPS) iMfs from nvAMD patients. Cytokines were added to the media based on the maximal level of each cytokine via ELISA of M(IFNγ and LPS)-derived media. Five (5) cytokines were tested (n=8; replicates for each cytokine): 0.25 ng/μl VEGF, 0.5 ng/μl IL-8, 2.5 ng/μl IL-6, 0.05 ng/μl IL-1β, and 0.8 5 ng/μl TNFα (PeproTech, Rocky Hill, N.J., USA). Anti-TNF (infliximab, 100 μg/μl Remsima, Celltrion Inc., South Korea) and anti-VEGF (Aflibercept, 40 μg/μl, Bayer Pharma AG, Berlin, Germany), with or without iMfs' supernatant were also studied in the CSA. Compounds' concentrations were based on previous studies (Grattendick et al., (2008)). Controls included medium-treated CSA and iMfs' supernatant treated CSA.

C57/BL6J 4-6 weeks old mice, treated in accordance with the guidelines of the Association for Research in Vision and Ophthalmology (ARVO), were utilized to generate the CSA. Experiments were conducted with the approval of the institutional animal care ethics committee. Five (5) minutes after injecting with ketamine, animals were evaluated for responses and euthanized by cervical dislocation. Eyes were immediately enucleated and kept in ice-cold ECGS medium containing 100 units/ml penicillin-streptomycin and 1% glutamine before dissection. A choroid-sclera complex from the mice was gently dissected along with retinal pigment epithelium (RPE). The complex was cut into 5-6 1 mm long pieces. Fragments were embedded in 30 μl of growth factor-reduced-Matrigel™ (BD Biosciences, Cat. 354230) in 24-well plates. The thickness of the Matrigel™ was approximately 0.4 mm. Plates were then incubated for 10 minutes in 37° C., in a 5% CO₂ cell culture incubator without medium to solidify the Matrigel™. Five hundred (500) μl of medium containing ECGM (C-22010, Promocell, Germany) 2.5% supplement mix (C-9215, Promocell, Germany), 5% FCS, 100 units/ml penicillin-streptomycin, and 1% glutamine, was added to each well. For the experimental groups containing the iMfs' supernatant, the medium was replaced with 250 μl of iMfs' supernatant. Medium for each well was changed every 3 days, and the cultures were fixed with 4% PFA after 8 days. Cultures were viewed with an inverted phase-contrast CKX41 Olympus microscope, and images were photographed with an Olympus DP70 digital camera (Olympus, Tokyo, Japan).

ImageJ software was used for sprouting area quantification. Sprouting area was selected by the software and measured after excluding the choroid tissue. Background (control well, for each plate, supplemented with medium only) was subtracted and analysis was performed for each sample. Ratios of each well of experimental group and its relative control from the same eye were calculated and averaged with its replicates.

### Laser-Induced Model of CNV (LI-CNV)

Long-Evans rats (8-12 weeks old) were used to generate LI-CNV. Animals were treated in accordance to the guidelines of ARVO. Experiments were conducted with the approval of the institutional animal care ethics committee. Before each procedure, rats were anesthetized by intraperitoneal injections of a mixture of 85% ketamine (Bedford Laboratories, Bedford, Ohio) and 15% xylazine (VMD, Arendonk, Belgium). Topical anesthesia (Oxybuprocaine HCL 0.4%; Fisher Pharmaceuticals, Tel-Aviv, Israel) was applied to each eye 10 minutes before intravitreal injections or laser photocoagulation.

Laser burns (5-7 burns per eye) were generated. Intravitreal injections of all compounds were performed using a PLI-100 Pico-Injector (Medical System Corp., Greenvale, N.Y.) two days following the laser to allow for CNV development, and every 2 days thereafter for a total of 10 days. iMfs were injected only once, two days after laser injury was performed.

Experimental groups included: 4 μl of PBS, 1 μl of 100 μg/μl infliximab, 1 μl of 100 μg/μl aflibercept, 1 μl of 100 μg/μl infliximab with 1×10⁵ M(IFNγ and LPS) suspended in 4 μl of PBS; 1 μl of 100 μg/μl aflibercept with 1×10⁵ M(IFNγ and LPS) suspended in 4 μl of PBS; and 1×10⁵ M(IFNγ and LPS) suspended in 4 μl of PBS, for a total of 5 experimental groups and 1 control group. Each experimental group included 8 injected eyes of 4 rats. The compound dosage was previously described (Xu et al., (2008)). Antibiotic ointment (5% Chloramphenicol) was applied after every injection. Choroid-RPE and retinal flat mounts were prepared 10 days following the laser injury.

### CNV Quantification

RPE-choroidal flat-mounts were fixated for one hour in 4% PFA and suspended overnight in isolectin solution (GS-Ib4 Alexa 594 staining solution, Molecular Probes, Eugene, Oreg.) containing 200 mM NaN₃ and 1 mM CaCl₂). Flat mounts were then washed 6 times for 20 minutes in PBS and embedded on a slide with a mounting medium. The CNV area around each laser injury was measured using the ImageJ software.

### Basic Statistical Analysis

Data was processed using the biostatistical package InStat (GraphPad, San Diego, Calif.). P<0.05 was considered to indicate statistical significance. Outliers were excluded (±2SD) from statistical analysis. Appropriate statistical tests were applied according to the results of a normalcy test and sample distribution and parameters.

### Macrophage Modulation Analysis

The inventors investigated the contribution of demographics (age, gender), disease status (nvAMD or control), and macrophage subtype (M0/M1/M2) on protein and mRNA expression profile and pro-angiogenic characteristics in-vivo and ex-vivo in the LI-CNV and CSA models, respectively. The analyses included protein quantification of PDGF, TNFα, VEGF, MCP1 (CCL2), and ICAM, as determined by ELISA, as well as quantification of gene expression levels of PDGF, TNFα, and VEGF, as determined by QPCR.

The inventors evaluated the contribution of patient (hereafter referred to as Cell Origin) vs. the lab manipulation of the iMfs (hereafter referred to as Environment) on macrophage protein and gene expression. To that end, a simple ANOVA using the statistical software R (https://cran.r-project.org/ and http://www.rstudio.com) was performed with the “reshape2” package. The amount of variance contributed by age, gender, disease status, and environment, and their interactions, and the unexplained variance was then calculated. This was performed using a mixed design ANOVA with repeated measures via R.

TABLE 1
Genes and factors associated with activated macrophages

| | Biomarker |
|---|---|
| No. | (Accession No.) |
| 1 | FOSB |
| | (NM_001114171) |
| 2 | TMEM176A |
| | (NM_018487.2) |
| 3 | TMEM176B |
| | (BC091471.1) |
| 4 | CCR2 |
| | (NM_001123041) |
| 5 | SLED1 |
| | (NC_000004.12) |
| 6 | OLR1 |
| | (NM_001172632) |
| 7 | MOP-1 |
| | (AB014771.1) |
| 8 | MS4A1 |
| | (NM_152866) |
| 9 | CD3G |
| | (NM_000073) |
| 10 | LEF1 |
| | (NM_001130713.2) |
| 11 | FAIM3 |
| | (NM_001142472.1) |
| 12 | SNORD116-24 |
| | (NC_000015.10) |
| 13 | GZMK |
| | (NM_002104) |
| 14 | KIR2DL3 |
| | (NM_015868.2) |
| 15 | SNORD116-8 |
| | (NC_000015.10) |
| 16 | SNORD116-15 |
| | (NC_000015.10) |
| 17 | IL7R |
| | (NM_002185) |
| 18 | SLAMF6 |
| | (NM_052931) |
| 19 | KLRK1 |
| | (NM_007360) |
| 20 | SNORD116-5 |
| | (NC_000015.10) |
| 21 | CD24 |
| | (NM_001291737) |
| 22 | SNORD116-14 |
| | (NC_000015.10) |
| 23 | KIR2DS2 |
| | (XM_017030275.2) |
| 24 | PRF1 |
| | (NM_005041) |
| 25 | TGFBR3 |
| | (NM_001195683) |
| 26 | CD2 |
| | (NM_001767) |
| 27 | KIR2DL1 |
| | (NM_014218) |
| 28 | FCER2 |
| | (NM_001207019) |
| 29 | KIR2DS4 |
| | (NM_012314) |
| 30 | SNORD116-3 |
| | (NC_000015.10) |
| 31 | ZFY |
| | (NM_001145275) |
| 32 | KIR2DL2 |
| | (NM_014219.2) |
| 33 | IKZF3 |
| | (NM_183232) |
| 34 | CST7 |
| | (NM_003650) |
| 35 | KLRB1 |
| | (NM_002258) |
| 36 | CD3D |
| | (NM_001040651) |
| 37 | SKAP1 |
| | (NM_001075099) |
| 38 | TCL1A |
| | (NM_001098725) |
| 39 | ITK |
| | (NM_005546) |
| 40 | ETS1 |
| | (NM_001143820) |
| 41 | P2RY10 |
| | (NM_014499) |
| 42 | CCR7 |
| | (NM_001838) |
| 43 | GPR56 |
| | (NM_001145770) |
| 44 | KIR2DS1 |
| | (NM_014512) |
| 45 | GZMB |
| | (NM_004131) |
| 46 | GZMA |
| | (NM_006144) |
| 47 | CD79A |
| | (NM_021601) |
| 48 | NKG7 |
| | (NM_005601) |
| 49 | CD8A |
| | (NM_001145873) |
| 50 | GNLY |
| | (NM_001302758) |
| 51 | SNORD94 |
| | (NC_000002.12) |
| 52 | CTSW |
| | (NM_001335.3) |
| 53 | PAX5 |
| | (NM_001280547) |
| 54 | SNORD116-20 |
| | (NC_000015.10) |
| 55 | FGFBP2 |
| | (NM_031950.3) |
| 56 | FCRLA |
| | (NM_001184866) |
| 57 | RNU5E |
| | (NR_002754.2) |
| 58 | SNORD116-1 |
| | (NC_000015.10) |
| 59 | SNORD116-17 |
| | (NC_000015.10) |
| 60 | FCRL1 |
| | (NM_001159397) |
| 61 | SH2D1A |
| | (NM_002351) |
| 62 | CD5 |
| | (NM_014207) |
| 63 | SPOCK2 |
| | (NM_001134434) |
| 64 | CD28 |
| | (NM_001243077) |
| 65 | CD22 |
| | (NM_024916) |
| 66 | KIR3DL2 |
| | (NM_001242867) |
| 67 | SNORA20 |
| | (NC_000006.12) |
| 68 | KIR3DS1 |
| | (NM_001083539.2) |
| 69 | RASGRP1 |
| | (NM_001128602) |
| 70 | CD247 |
| | (NM_000734) |
| 71 | SAMD3 |
| | (NP_001017373.2) |
| 72 | CD96 |
| | (NP_005807.1) |
| 73 | NFATC2 |
| | (NM_001136021) |
| 74 | RHOH |
| | (NM_001278359) |
| 75 | FCRL6 |
| | (NP_001004310.2) |
| 76 | FCRL3 |
| | (NM_001024667) |
| 77 | IFITM1 |
| | (NM_003641) |
| 78 | BTLA |
| | (NM_001085357) |
| 79 | IL2RB |
| | (NM_000878) |
| 80 | PDGF |
| | (AAB26566.1) |
| 81 | TNFα |
| | (NM_000594) |
| 82 | VEGF |
| | (AAP86646.1) |
| 83 | MCP1 (CCL2) |
| | (NM_002982) |
| 84 | ICAM |
| | (NM_000201) |

### Example 1

### Exogenous TNFα Enhances, and Anti-TNF Therapy Abolishes Pro-Angiogenic Effect of Human Macrophages on Choroid Sprouting Ex Vivo

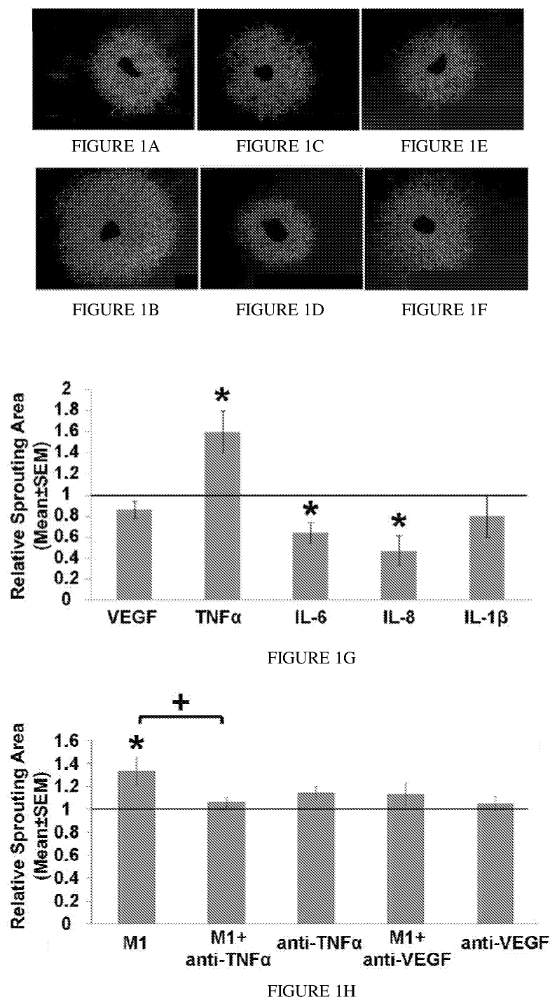

TNFα was found to be associated with enhanced choroidal sprouting in CSA as compared with control (n=8, Mean of relative ratio between treated and untreated wells±SEM>1.6±0.2, p=0.01, t-test). In contrast, a decreased sprouting area was found to be associated with IL-6 (n=9, 0.64±0.1, p=0.01) and IL-8 (n=8, 0.47±0.14, p=0.007). IL-1β (n=10, 0.8±0.2, p=0.3) and VEGF (n=9, 0.86±0.08, p=0.16) did not affect the sprouting area (FIG. 1G). The lack of an effect following VEGF supplementation in this model may be due to the CSA growth medium itself contains high levels of VEGF, which may nullify the effect of the supplemented VEGF. The enhanced CNV growth following the addition of M(IFNγ and LPS) supernatant, but not VEGF alone, could indicate that iMfs support CNV in a non-VEGF dependent pathway.

The addition of an anti-TNF compound abolished the contribution of the iMfs' supernatant to the choroidal sprouting growth (M(IFNγ and LPS)s' supernatant+anti-TNF n=7, Mean of relative ratio between treated and untreated wells±SEM>1.06±0.04, t-test: p=0.2; M(IFNγ and LPS): n=7, 1.33±0.12, t-test: p=0.04; paired t-test of anti-TNF+M(IFNγ and LPS)s' supernatant compared to M(IFNγ and LPS)s' supernatant alone: p=0.03). Anti-TNF by itself did not affect sprouting area (n=7, 1.14±0.06, p=0.06, t-test) (FIGS. 1A-1D and 1H). These findings suggest that TNFα mediates the pro-angiogenic effect of iMfs in this model.

### Example 2

### Anti-TNF Therapy Abolishes Pro-Angiogenic Effect of Human Macrophages In-Vivo

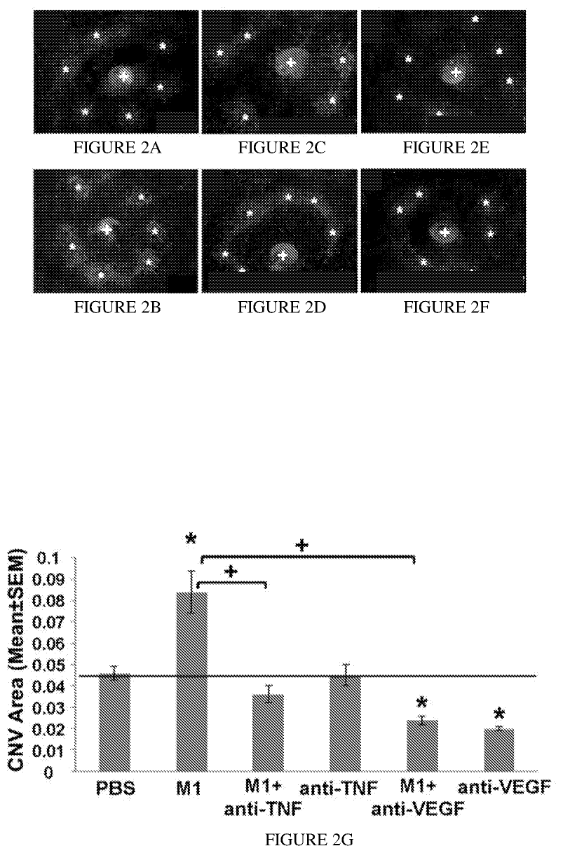

Intravitreal injections of anti-TNF abolished the contribution of M(IFNγ and LPS) iMfs to the CNV in-vivo (anti-TNF+M(IFNγ and LPS): n=8, Mean of CNV size±SEM>0.036±0.004, t-test as compared to PBS injected group: p=0.13; M(IFNγ and LPS): n=6, 0.084±0.01, t-test compared to PBS: p=0.007, t-test of anti-TNF+M(IFNγ and LPS) compared to M(IFNγ and LPS): p=0.0008). Consistent with the ex-vivo findings, anti-TNF by itself had no effect on CNV growth (n=8, 0.045±0.005, p=0.77) (FIGS. 2A-2D and 2G).

Intravitreal injections of an anti-VEGF compound with or without iMfs abolished CNV growth (anti-VEGF compared to PBS injected groups: n=7, 0.024±0.002, p=0.003; anti-VEGF with M(IFNγ and LPS) compared to PBS: n=8, 0.02±0.001, p=0.003; anti-VEGF as compared to anti-VEGF with M(IFNγ and LPS): p=0.12; anti-VEGF with M(IFNγ and LPS) as compared to M(IFNγ and LPS): p=0.0002) (FIGS. 2A-2B and 2E-2G).

### Example 3

### TNFα Gene and Protein Expression in Macrophages are Largely Modifiable

Via statistical analysis it was evident that TNFα gene and protein expression are largely modifiable. According to ANOVA, 29% of TNFα, 29% of VEGF and 29% of PDGF gene expression of the 3 macrophage subtypes tested in the cell culture of nvAMD patients (n=7) and age-matched controls (n=9), were explained by the cell origin, i.e., patient derived factors. Sixteen (16) %, 20% and 9% of TNFα, VEGF and PDGF gene expression, respectively, were explained by the environment (cell culture manipulations to generate macrophage phenotype), while 55%, 51% and 61% of TNFα, VEGF and PDGF gene expression, respectively, were unexplained.

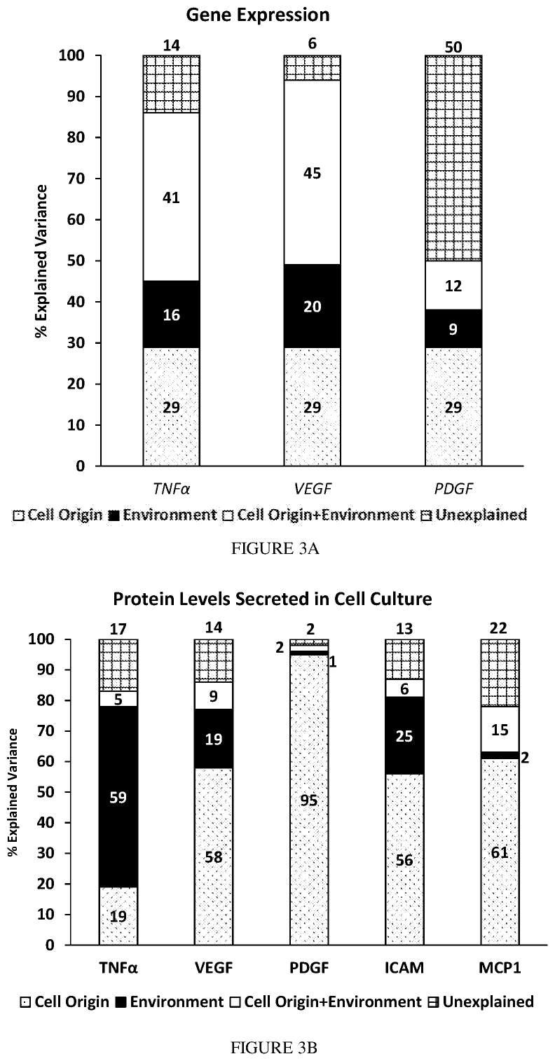

Using the mixed designed ANOVA with repeated measures, the inventors added the non-modifiable factors of age, gender and disease status to the cell origin. According to this model, 58%, 65% and 21% of TNFα, VEGF and PDGF gene expression, respectively, were explained by the environment and its interactions with patients' factors. The unexplained variance of TNFα, VEGF and PDGF gene expression, decreased to 14%, 6% and 50%, respectively (FIG. 3A).

In addition, only 19% of TNFα protein levels' variability in a cell culture (n=13 nvAMD patients and 13 age-matched controls), were explained by cell origin, whereas 64% were explained by environment, and its interactions with the patients' factors. Protein levels of other cytokines including VEGF, PDGF, ICAM and MCP1 were found to be less dependent on modifiable factors, with only 28%, 4%, 31% and 17% of the variability explained by environment and its interaction, respectively. Unlike TNFα, a relatively high percentage of the protein levels' variability of these cytokines was explained by the cell origin (VEGF: 58%; PDGF: 95%; ICAM: 56%; MCP1: 61%) (FIG. 3B).

### Example 4

### Macrophages' Function in Experimental CNV In-Vivo and Ex-Vivo are Modifiable

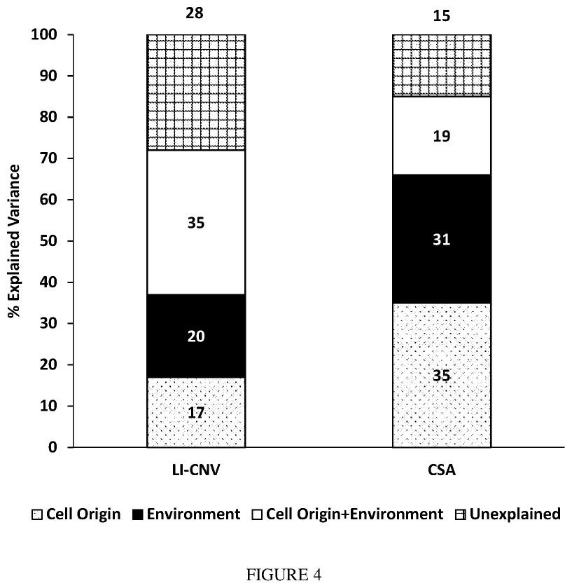

Using the mixed design ANOVA with repeated measures, the inventors were able to find that the function of iMfs in the ex-vivo and in-vivo models is modifiable. Only 17% of the variability of the 3 macrophage subtypes' effect on LI-CNV were explained by cell origin (n=8 nvAMD patients and 9 controls), whereas 55% were explained by environment and its interactions. Only 35% of the variability of the 3 macrophage subtypes' effect on the CSA, were explained by cell origin, and 55% were explained by the modifiable Environment (FIG. 4).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating a retinal disease in a subject, the method comprising:
i. determining at least one parameter selected from gene expression or factor secretion levels of one or more biomarkers listed under Table 1, in a sample obtained from the subject; and
ii. administering to a subject having an alteration of at least one parameter relative to control, a pharmaceutical composition comprising a therapeutically effective amount of a macrophage modulating compound and at least one pharmaceutically acceptable carrier or diluent.

2. The method of claim 1, wherein said macrophage modulating compound has increased anti-angiogenic activity.

3. The method of claim 1, wherein said retinal disease is neovascular age related macular degeneration (nvAMD).

4. The method of claim 3, wherein said nvAMD comprises choroidal neovascularization (CNV).

5. The method of claim 1, wherein said alteration is an increased expression of at least 1.5-fold of a biomarker selected from the group consisting of: FOSB, TMEM176A, TMEM176B, SLED1, CCR2, OLR1 and MOP-1, compared to control, and is indicative of said subject is having a state suitable for treatment by a macrophage modulating compound.

6. The method of claim 1, wherein said alteration is a decreased expression of at least 1.5-fold of a biomarker selected from the group consisting of: MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA and IL2RB, compared to control, and is indicative of said subject is having a state suitable for treatment by a macrophage modulating compound.

7. The method of claim 1, wherein said biomarker is selected from the group consisting of: PDGF, TNFα, VEGF, MCP1 (CCL2), and ICAM.

8. The method of claim 1, wherein increased secretion of at least 20% of said biomarker, compared to control, is indicative of said subject is having a state suitable for treatment by a macrophage modulating compound.

9. The method of claim 1, wherein said macrophage modulating compound inhibits predominantly activated macrophages.

10. The method of claim 1, wherein said macrophage modulating compound is a tumor necrosis factor alpha (TNFα) inhibitor.

11. The method of claim 10, wherein said TNFα inhibitor is selected from the group consisting of: nucleic acids, peptides, polypeptides, peptidomimetics, carbohydrates, lipids or other organic or inorganic molecules.

12. The method of claim 10, wherein said TNFα inhibitor is selected from the group consisting of: etanercept, infliximab, adalimumab, golimumab and certolizumab.

13.-25. (canceled)

26. A kit for determining macrophage activation in a sample, comprising: at least one molecule that binds to a target biomarker selected from the group consisting of: FOSB, TMEM176A, TMEM176B, CCR2, SLED1, OLR1, MOP-1, MS4A1, CD3G, LEF1, FAIM3, SNORD116-24, GZMK, KIR2DL3, SNORD116-8, SNORD116-15, IL7R, SLAMF6, KLRK1, SNORD116-5, CD24, SNORD116-14, KIR2DS2, PRF1, TGFBR3, CD2, KIR2DL1, FCER2, KIR2DS4, SNORD116-3, ZFY, KIR2DL2, IKZF3, CST7, KLRB1, CD3D, SKAP1, TCL1A, ITK, ETS1, P2RY10, CCR7, GPR56, KIR2DS1, GZMB, GZMA, CD79A, NKG7, CD8A, GNLY, SNORD94, CTSW, PAX5, SNORD116-20, FGFBP2, FCRLA, RNU5E, SNORD116-1, SNORD116-17, FCRL1, SH2D1A, CD5, SPOCK2, CD28, CD22, KIR3DL2, SNORA20, KIR3DS1, RASGRP1, CD247, SAMD3, CD96, NFATC2, RHOH, FCRL6, FCRL3, IFITM1, SNORA22, BTLA, IL2RB, PDGF, TNFα, VEGF, MCP1 (CCL2) and ICAM.

27. The kit of claim 26, wherein said molecule is selected from the group consisting of: a polynucleotide or a polypeptide.

28. The kit of claim 27, wherein said polynucleotide hybridizes to said target.

29. The kit of claim 27, wherein said polypeptide is an antibody.

30. The kit of claim 26, for determining suitability for treatment of a nvAMD disease in a subject by a TNFα inhibitor.

31. (canceled)
